(19) United States
(12) Patent Application Publication
(10) Pub. No.: US 2019/0049299 A1
(43) Pub. Date: Feb. 14, 2019

(54) Method for Rapidly Detecting Salmonella Typhimurium in Milk by Raman Microspectroscopy Based on Incorporation of Heavy Water

(71) Applicant: Jiangnan University, Wuxi (CN)

(72) Inventors: Yunfei Xie, Wuxi (CN); Jingjing Feng, Wuxi (CN); Weirong Yao, Wuxi (CN); Yahui Guo, Wuxi (CN); Yuliang Cheng, Wuxi (CN); He Qian, Wuxi (CN)

(73) Assignee: Jiangnan University

(21) Appl. No.: 15/815,341

(22) Filed: Nov. 16, 2017

(30) Foreign Application Priority Data
Aug. 10, 2017 (CN) 201710680746.5

(51) Int. Cl. G01J 3/44; G01N 15/02; G01N 21/65; C12Q 1/04; G01N 33/04

(52) U.S. Cl. CPC G01J 3/44; G01N 15/0211; G01N 33/04; C12Q 1/04; G01N 21/65

(57) ABSTRACT

A method for rapidly detecting S. typhimurium in milk through a Raman spectrum based on heavy water marking. The method comprises the steps of adding to-be-detected milk possibly containing S. typhimurium into culture media containing heavy water for culturing, then collecting cultured S. typhimurium cells and conducting Raman spectrum scanning, and analyzing and processing an obtained original Raman spectrum, so that S. typhimurium in the to-be-detected milk is detected. According to the method of the invention, D₂O marking is adopted, and the Raman spectrum is used for rapidly detecting S. typhimurium in milk, so that the method has the advantages of being short in detection time (4-8 h), high in sensitivity, low in cost, easy to operate, convenient and fast to implement and the like; the detection limit is 10⁴-10⁸ cfu/mL, interference from the matrix in a milk sample is small, and the method is an ideal method for rapidly detecting S. typhimurium, has extremely good actual application prospects, and is suitable for being widely applied in the fields of food safety, environment monitoring and the like.

### BACKGROUND OF THE INVENTION

### Technical Field

The invention relates to a method for detecting S. typhimurium in milk, in particular to a method for rapidly detecting S. typhimurium in milk through a Raman spectrum based on incorporation of heavy water (D₂O), and belongs to the technical field of pathogenic bacterium detection.

### Description of Related Art

As a common food-borne pathogenic bacterium, S. typhimurium is aerobic, facultative anaerobic and spore-free, and belongs to gram negative enterobacteriaceae. Nearly one hundred of types of S. typhimurium (bacterial strains) have been found. The S. typhimurium can grow in an extremely wide temperature range (10-42 DEG C.), the optimal temperature is 37 DEG C., and the optimal pH value is 6.8-7.8. S. typhimurium is widely distributed, and certain salmonella can cause diseases in humans, livestock and wild animals and be important pathogenic bacteria causing food poisoning of humans. According to the statistics, food poisoning caused by salmonella is the top of bacterial food poisoning in all countries in the world in recent years and is also the top in inland areas in China, and severe health hazards and economic losses of people are caused by S. typhimurium. At present, the food safety problem is outstanding, and particularly, the food safety problem caused by food-borne pathogenic bacteria draws the attention of national governments and institutions for academic research.

Most conventional S. typhimurium detection methods in China are traditional microorganism culturing methods, and the method mainly comprises the steps of pre-propagation of bacteria (8-18 h), propagation of bacteria (18-24 h), plate culture separation (18-24), biochemical testing, serological identification and the like. By adoption of the methods, results are accurate and reliable, however, operation is complex, time consumption is high, various reagents are required, and consequentially, the methods cannot meet the current requirement for detection of food-borne pathogenic bacteria any more.

### BRIEF SUMMARY OF THE INVENTION

The invention mainly aim to provide a method for rapidly detecting salmonella in milk through a Raman spectrum based on heavy water marking, and the method has the characteristics of being easy and convenient to operate, fast and convenient to implement and the like and thus overcomes the defects in the prior art.

For achieving the above purpose, the following technical scheme is adopted by the invention.

The embodiment of the invention provides a method for rapidly detecting S. typhimurium in milk through a Raman spectrum based on heavy water marking, and the method comprises the steps of:

adding to-be-detected milk possibly containing S. typhimurium into culture media containing heavy water for culturing, then collecting cultured salmonella cells and conducting Raman spectrum scanning, and analyzing and processing an obtained original Raman spectrum, so that salmonella in the to-be-detected milk is detected.

In certain specific embodiments, if salmonella exists in the to-be-detected milk, a CD peak appears in the corresponding original Raman spectrum within the range of 2040 cm⁻¹-2300 cm⁻¹.

In certain specific embodiments, the method comprises the steps of:

(1) adding a series of S. typhimurium seed solutions with different concentrations into the culture media containing heavy water for culturing for the log phase, then collecting S. typhimurium cells at different time points respectively, conducting Raman spectrum scanning, and analyzing and processing an obtained Raman spectrum, so that a standard curve of CD/(CD+CH) % and the log value of the original S. typhimurium concentration is obtained, wherein CD/(CD+CH) % is the ratio of the peak height of the CD peak to the sum of the peak heights of the CD peak and the CH peak in the Raman spectrum;

(2) adding the to-be-detected milk possibly containing S. typhimurium into the culture media containing heavy water for culturing for the log phase, then collecting S. typhimurium cells at different time points, conducting Raman spectrum scanning, and analyzing and processing an obtained Raman spectrum and comparing the obtained Raman spectrum with the standard curve, so that the concentration of S. typhimurium in the to-be-detected milk is measured.

In certain specific embodiments, the step (2) further comprises the sub-step of setting operating parameters of a laser Raman spectrometer, wherein the operating parameters include the wavelength of exciting light, the laser power and the scanning time.

Preferably, the wavelength of the exciting light is set to 532 nm, the laser power is set to 8 mw, and the scanning time is set to 10 s.

Compared with the prior art, the invention is based on D₂O marking and adopts the Raman spectrum for rapidly detecting S. typhimurium in milk, so that the method has the advantages of being short in detection time (4-8 h), high in sensitivity, low in cost, easy to operate, convenient and fast to implement and the like; the detection limit is 10⁴-10⁸ cfu/mL, interference from the matrix in a milk sample is small, and the method is an ideal method for rapidly detecting salmonella, has extremely good actual application prospects, and is suitable for being widely applied in the fields of food safety, environment monitoring and the like.

### BRIEF DESCRIPTION OF THE SEVERAL VIEWS OF THE DRAWINGS

FIGS. 1a-1f respectively show growth curves of S. typhimurium in different culture media and corresponding Raman spectrums in one typical embodiment of the invention.

FIGS. 2a-2c respectively show linear relation curve charts of CD/(CD+CH) % and the Log value of the added S. typhimurium concentration when S. typhimurium is cultured in culture media with the D2O content of 50% for 4 h, 6 h and 8 h respectively in one typical embodiment of the invention.

FIG. 3a and FIG. 3b respectively show a Raman spectrum chart and a linear relation curve chart of CD/(CD+CH) % and the Log value of the added S. typhimurium concentration when salmonella in the to-be-detected milk is placed in culture media prepared from D₂O with the concentration of 50% in one typical embodiment of the invention.

### DETAILED DESCRIPTION OF THE INVENTION

As mentioned above, for overcoming the defects of the prior art, the inventor put forwards the technical scheme of the invention through long-term study and mass practices, and a method for rapidly detecting S. typhimurium in milk through a Raman spectrum based on heavy water marking is mainly provided. In general, the method comprises the steps of adding to-be-detected milk into culture media containing heavy water, then collecting cultured S. typhimurium cells and conducting Raman spectrum scanning, and judging whether S. typhimurium exists or not according to the fact whether an obvious C-D peak appears within the range of 2040 cm⁻¹-2300 cm⁻¹. The method is short in detection time (4-8 h) and has the advantages of being high in sensitivity, low in cost, easy to operate, convenient and fast to implement and the like, the detection limit is 10⁴-10⁸ cfu/mL, and S. typhimurium in milk can be detected rapidly. A further explanation of the technical scheme, the implementation process and principle of the technical scheme and the like is given as follows.

On one aspect of the technical scheme of the invention, a method for rapidly detecting salmonella in milk through a Raman spectrum based on heavy water marking comprises the steps of:

adding to-be-detected milk possibly containing S. typhimurium into culture media containing heavy water for culturing, then collecting cultured S. typhimurium cells and conducting Raman spectrum scanning, and analyzing and processing an obtained original Raman spectrum, so that S. typhimurium in the to-be-detected milk is detected.

In certain specific embodiments, if S. typhimurium exists in the to-be-detected milk, a CD peak appears in the corresponding original Raman spectrum within the range of 2040 cm⁻¹-2300 cm⁻¹

In certain specific embodiments, the method comprises the steps of:

(1) adding a series of S. typhimurium seed solutions with different concentrations into the culture media containing heavy water for culturing for the log phase, then collecting S. typhimurium cells at different time points respectively, conducting Raman spectrum scanning, and analyzing and processing an obtained Raman spectrum, so that a standard curve of CD/(CD+CH) % and the log value of the original S. typhimurium concentration is obtained, wherein CD/(CD+CH) % is the ratio of the peak height of the CD peak to the sum of the peak heights of the CD peak and the CH peak in the Raman spectrum;

(2) adding the to-be-detected milk possibly containing S. typhimurium into the culture media containing heavy water for culturing for the log phase, then collecting S. typhimurium cells at different time points, conducting Raman spectrum scanning, and analyzing and processing an obtained Raman spectrum and comparing the obtained Raman spectrum with the standard curve, so that the concentration of S. typhimurium in the to-be-detected milk is measured.

In certain specific embodiments, the step (2) further comprises the sub-step of setting operating parameters of a laser Raman spectrometer, wherein the operating parameters include the wavelength of exciting light, the laser power and the scanning time.

Preferably, the wavelength of the exciting light is set to 532 nm, the laser power is set to 8 mw, and the scanning time is set to 10 s.

The culturing temperature is 37 DEG C., and the culturing time is 4-8 h.

Preferably, the S. typhimurium detection limit of the method is 10⁴-10⁸ cfu/mL.

Preferably, the concentrations of the S. typhimurium seed solutions are 10⁴ CFU/mL, 5×10⁴ CFU/mL, 10⁵ CFU/mL, 5×10⁵ CFU/mL, 10⁶ CFU/mL, 5×10⁶ CFU/mL, 10⁷ CFU/mL, 5×10⁷ CFU/mL and 10⁸ CFU/mL respectively.

In certain specific embodiments, S. typhimurium cultured in the culture media containing heavy water has the C-D peak at the position with the Raman shift of 2040 cm⁻¹˜2300 cm⁻¹.

In certain specific embodiments, in the standard curve, CD/(CD+CH) % is in the linear relation with the Log value of the original S. typhimurium concentration.

Specifically, when S. typhimurium is cultured for 4 h, the linear relation is: y=2.7753x−14.377, R²=0.9894, wherein x is the Log value of the original S. typhimurium concentration, and y is CD/(CD+CH) %.

When S. typhimurium is cultured for 6 h, the linear relation is: y=1.8209x−5.1865, R²=0.963, wherein x is the Log value of the original S. typhimurium concentration, and y is CD/(CD+CH) %.

When S. typhimurium is cultured for 8 h, the linear relation is: y=0.9491x+2.6985, R²=0.9889, wherein x is the Log value of the original S. typhimurium concentration, and y is CD/(CD+CH) %

In certain specific embodiments, the step (2) comprises the sub-step of analyzing and processing the obtained Raman spectrum and substituting the obtained CD/(CD+CH) % to the standard curve obtained in the step (1), so that the added recovery is calculated.

Preferably, the content of heavy water in the culture media containing heavy water is 0˜100 wt % and preferably is 50 wt %.

Preferably, a preparation method of the S. typhimurium seed solutions comprises the steps of placing S. typhimurium in sterilized ultrapure water culture media for activation, and obtaining the S. typhimurium seed solutions at the later stage in the process of culturing the S. typhimurium for the log phase.

In certain specific embodiments, the method for rapidly detecting S. typhimurium in milk through the Raman spectrum based on heavy water marking can comprise the following steps of:

(1) Preparation of culture media, specifically,

nutrient broth solid culture media are weighed and placed in heavy water, and the nutrient broth solid culture media are placed in a high-pressure sterilizing pot at the temperature of 121 DEG C. so as to be sterilized for 20 min for later use after being evenly oscillated and dissolved.

(2) Activation and marked culture of S. typhimurium, specifically:

a. S. typhimurium is obtained through sterile operation and placed in the sterilized ultrapure water nutrient broth culture media so as to be activated, and S. typhimurium seed solutions are obtained at the later stage in the process of culturing salmonella for the log phase for later use;

b. growth curve: the S. typhimurium seed solutions are added into the nutrient broth culture media prepared from heavy water so as to be cultured, the absorbance values OD₆₀₀ of S. typhimurium at different time points within 0-24 h in the culturing process are measured respectively, a growth curve is drawn based on the absorbance values OD₆₀₀ and the time, and meanwhile, S. typhimurium cells are centrifugally collected at different time points and placed on tin foil paper.

c. the S. typhimurium seed solutions are diluted to different concentrations including 10⁴ CFU/mL, 5×10⁴ CFU/mL, 10⁵ CFU/mL, 5×10⁵ CFU/mL, 10⁶ CFU/mL, 5×10⁶ CFU/mL, 10⁷ CFU/mL, 5×10⁷ CFU/mL and 10⁸ CFU/mL and are added into the nutrient broth culture media prepared from heavy water so as to be cultured respectively; S. typhimurium with different concentrations is cultured for the log phase, and S. typhimurium cells are centrifugally collected at different time points in the log phase and placed on tin foil paper.

(3) Raman spectrum detection: Raman spectrum testing is conducted on the S. typhimurium cells placed on the tin foil paper in the sub-steps b, c of step (2); parallel-point testing is conducted on each sample ten times.

Wherein, measurement conditions of a Raman spectrometer in steps (3) are set as: the detection wavelength of a He—Ne laser device is 532 nm, the laser power is about 8 mW, and the scanning time is 10 s.

Wherein, CD/(CD+CH) % refers to the ratio of the peak height of the CD peak to the sum of the peak heights of the CD peak and the CH peak in the Raman spectrum.

(4) Data processing, specifically,

a Raman spectrum detection result is analyzed, and the peak height of the CD peak and the peak height of the CH peak are recorded; the linear relation between the CD/(CD+CH) % and the Log value of the original S. typhimurium concentration is established, a standard curve is obtained, and thus quantitative detection of S. typhimurium is conducted according to Raman signals.

(5) The process of actually applying the method of the invention to detection of S. typhimurium in pasteurized milk specifically comprises the following steps that:

the S. typhimurium seed solutions are added into the pasteurized milk (no bacterial colony is detected through the bacterial colony counting method), and S. typhimurium, with the final concentrations of 10⁴ CFU/mL, 5×10⁴ CFU/mL, 10⁵ CFU/mL, 5×10⁵ CFU/mL, 10⁶ CFU/mL, 5×10⁶ CFU/mL, 10⁷ CFU/mL, 5×10⁷ CFU/mL and 10⁸ CFU/mL respectively, in the milk is added into D₂O with the concentration of 50% so as to be cultured respectively; S. typhimurium cells are centrifugally collected at different time points in the log phase and placed on tin foil paper, and Raman spectrum testing is conducted; parallel-point testing is conducted on each sample ten times, and the peak height of the CD peak and the peak height of the CH peak are recorded; CD/(CD+CH) % is substituted into the standard curve obtained above, and the added recovery is calculated.

(6) The process of actually applying the method of the invention to detection of S. typhimurium in to-be-detected milk specifically comprises the following steps that:

the to-be-detected milk containing S. typhimurium are added into the culture media containing D₂O with the concentration of 50% for so as to be cultured for the log phase, then S. typhimurium cells are collected at different time points, Raman spectrum scanning is conducted, and an obtained Raman spectrum is analyzed and processed and compared with the standard curve, so that the concentration of S. typhimurium in the to-be-detected milk is measured.

A further detailed description of the technical scheme of the invention is given with embodiments and accompanying drawings as follows, however, the invention is not only limited to the following embodiments.

In the following embodiment, the following detection conditions are adopted.

Parameters of the laser Raman spectrometer are set as: the detection wavelength of the He—Ne laser device is 532 nm, the laser power is about 8 mw, and the scanning time is 10 s.

Of course, those skilled in the art can also set other parameters of the laser Raman spectrometer according to learning materials.

After the detection conditions are determined, S. typhimurium in milk can be detected according to the following method:

(1) Preparation of culture media, specifically,

0.9 g of nutrient broth solid culture media are weighed and placed in 50 mL of ultrapure water, D₂O with the concentration of 50% and D₂O with the concentration of 100% respectively, and the nutrient broth solid culture media are placed in a high-pressure sterilizing pot at the temperature of 121 DEG C. so as to be sterilized for 20 min for later use after being evenly oscillated and dissolved

(2) Activation and marked culture of S. typhimurium, specifically,

a. S. typhimurium is obtained through sterile operation and placed in the sterilized ultrapure water nutrient broth culture media so as to be activated, and S. typhimurium seed solutions are obtained at the later stage in the process of culturing S. typhimurium for the log phase for later use;

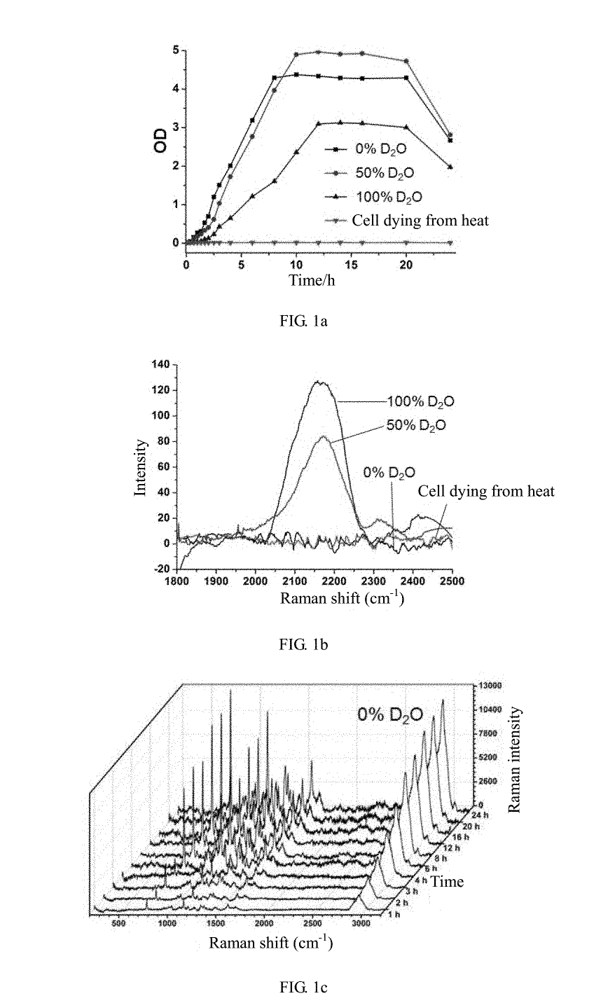

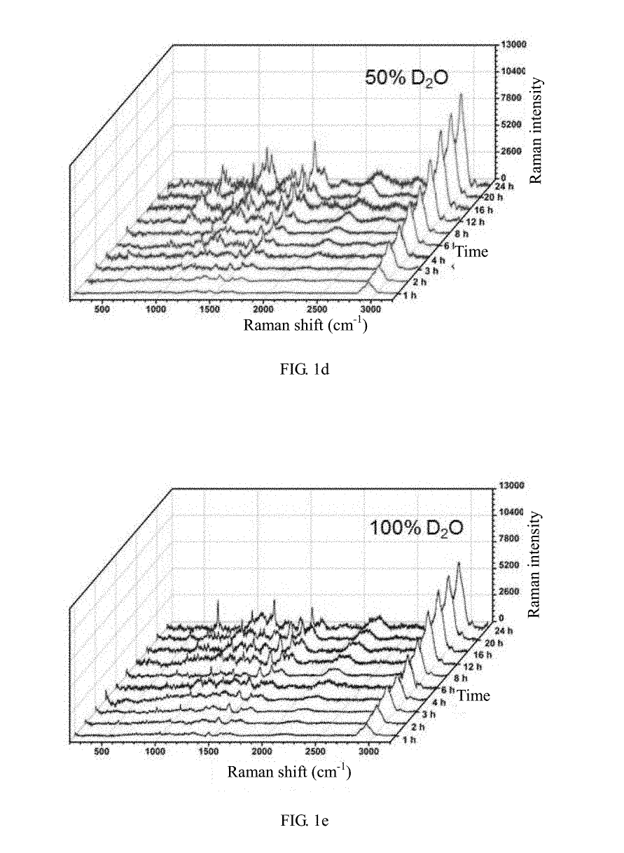

b. growth curve: the S. typhimurium seed solutions are added into the nutrient broth culture media prepared from the ultrapure water (serving as the comparison group), the D₂O with the concentration of 50% and the D₂O with the concentration of 100% so as to be cultured, and the S. typhimurium seed solutions are placed in nutrient broth prepared from D₂O with the concentration of 50% after being sterilized for 20 min at the temperature of 121 DEG C. so as to be cultured in the same way; the absorbance values OD₆₀₀ of S. typhimurium at different time points within 0-24 h in the culturing process are measured respectively, a growth curve is drawn based on the absorbance values OD₆₀₀ and the time, and as is shown in FIGS. 1a-1f, the result shows that the state of the S. typhimurium cultured in the D₂O with the concentration of 50% is not obviously affected; meanwhile, S. typhimurium cells are centrifugally collected at different time points and placed on tin foil paper.

In the method, salmonella is cultured through the ultrapure water and heavy water marking, and Raman spectrum testing is conducted on the obtained S. typhimurium cells under the wavelength of 532 nm. For the S. typhimurium cultured in heavy water, an obvious C-D peak appears within 2040 cm⁻¹-2300 cm⁻¹, and for the S. typhimurium cultured with the ultrapure water, no C-D peak appears. When the salmonella is cultured in the culture media prepared from D₂O with the concentration of 50%, an obvious C-D peak appears in 1 h, and then it can be confirmed that S. typhimurium exists.

c. the S. typhimurium seed solutions are diluted to different concentrations including 10⁴ CFU/mL, 5×10⁴ CFU/mL, 10⁵ CFU/mL, 5×10⁵ CFU/mL, 10⁶ CFU/mL, 5×10⁶ CFU/mL, 10⁷ CFU/mL, 5×10⁷ CFU/mL and 10⁸ CFU/mL and are added into the nutrient broth culture media prepared from D₂O with the concentration of 50% so as to be cultured respectively; S. typhimurium with different concentrations is cultured for the log phase, and S. typhimurium cells are centrifugally collected at different time points in the log phase and placed on tin foil paper.

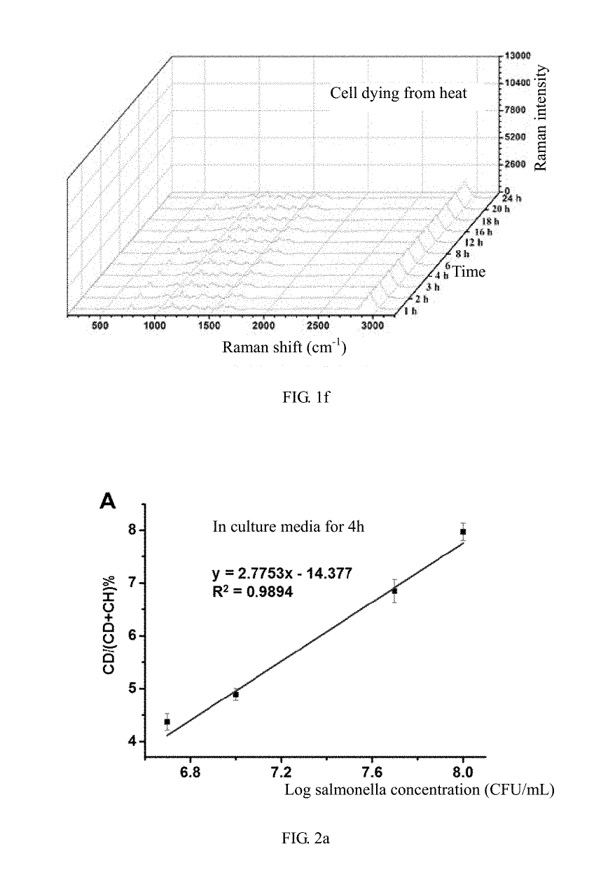

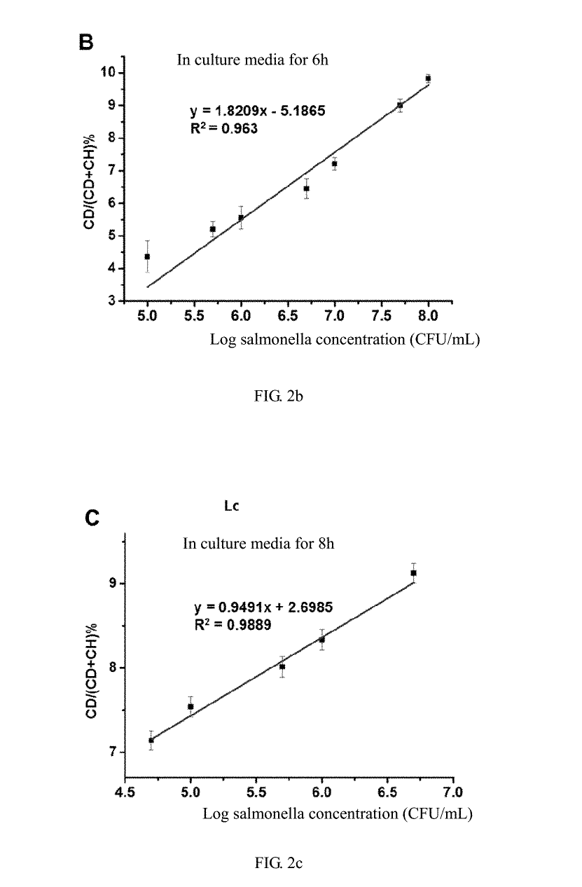

(3) Raman spectrum detection: Raman spectrum testing is conducted on the S. typhimurium cells placed on the tin foil paper in the sub-steps b, c of step (2). Parallel-point testing is conducted on each sample ten times. A Raman spectrum detection result is analyzed, and the peak height of the CD peak and the peak height of the CH peak are recorded. The result shows that when S. typhimurium is cultured for 1 h, both for the S. typhimurium cultured in the culture media prepared from D₂O with the concentration of 50% and for the S. typhimurium cultured with the culture media prepared from D₂O with the concentration of 100%, obvious C-D peaks occur, however, for the S. typhimurium cultured in the ultrapure water, no C-D peak appears in the Raman spectrum. For the S. typhimurium sterilized at a high temperature and cultured in the culture media, no C-D peak appears either. By analyzing the testing result in the sub-step (2)c, it is found that in the log phase, CD/(CD+CH) % has a good linear relation with the Log value of the original S. typhimurium concentration, a standard curve is drawn, quantitative detection of S. typhimurium is achieved through Raman signals accordingly, and the result is shown in FIGS. 2a-2c.

(4) The process of applying the method for rapidly detecting S. typhimurium in milk through the Raman spectrum based on heavy water marking to detection of S. typhimurium in pasteurized milk specifically comprises the following steps that:

the S. typhimurium seed solutions are added into the pasteurized milk (no bacterial colony is detected through the bacterial colony counting method), and salmonella, with the final concentrations of 10⁴ CFU/mL, 5×10⁴ CFU/mL, 10⁵ CFU/mL, 5×10⁵ CFU/mL, 10⁶ CFU/mL, 5×10⁶ CFU/mL, 10⁷ CFU/mL, 5×10⁷ CFU/mL and 10⁸ CFU/mL respectively, in the milk is added into D₂O with the concentration of 50% so as to be cultured respectively;

the to-be-detected milk containing S. typhimurium is into the culture media containing the D₂O with the concentration of 50% so as to be cultured for the log phase;

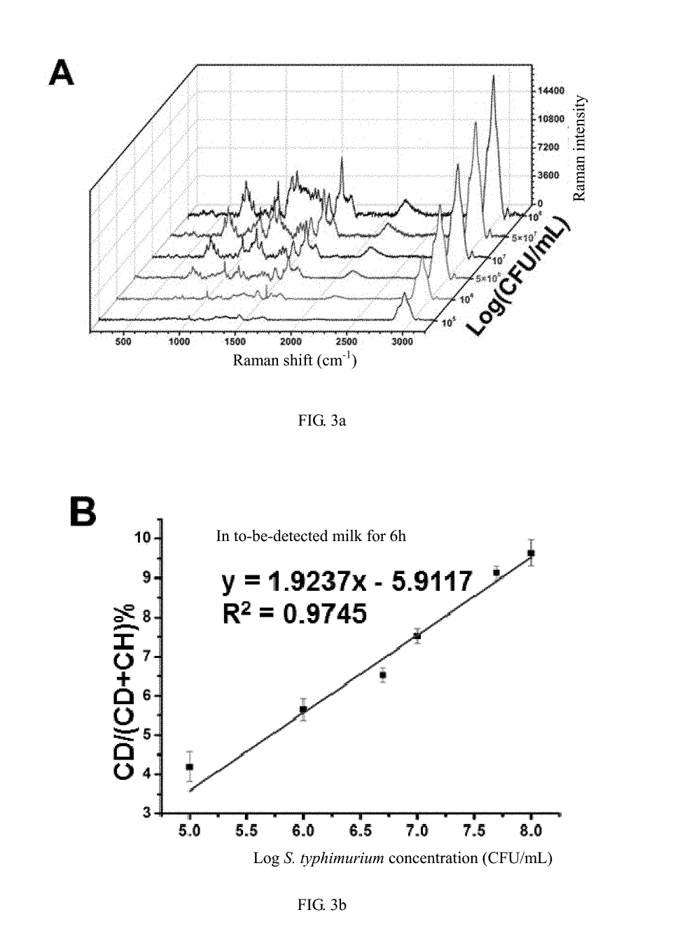

S. typhimurium cells are centrifugally collected at different time points in the log phase and placed on tin foil paper, and Raman spectrum testing is conducted; parallel-point testing is conducted on each sample ten times, and the peak height of the CD peak and the peak height of the CH peak are recorded, and the result is shown in FIG. 3a and FIG. 3b. The result shows that in the milk, CD/(CD+CH) % is also in a good linear relation with the Log value of the original S. typhimurium concentration. The S. typhimurium with different added concentrations is cultured for different times, the added recovery calculated according to the formula of added recovery=Log N₂/Log N₁% is 89.7%-104.61%, and the result is shown in FIG. 1.

TABLE 1
Added recovery obtained when S. typhimurium with different
added concentrations is cultured for different times

| | Added | Detection | |
|---|---|---|---|
| Time | Concentration | Concentration | Recovery |
| (h) | Log N₁ | Log N₂ | (%) |
| 4 | 8 | 7.725 ± 0.098 | 96.56 |
| 4 | 7.699 | 7.597 ± 0.109 | 98.67 |
| 6 | 5 | 5.149 ± 0.12 | 103.0 |
| 6 | 6 | 5.953 ± 0.078 | 99.21 |
| 6 | 7 | 6.978 ± 0.19 | 99.69 |
| 6 | 8 | 8.141 ± 0.28 | 101.8 |
| 8 | 5 | 4.865 ± 0.191 | 97.30 |
| 8 | 5.699 | 5.112 ± 0.125 | 89.70 |
| 8 | 6 | 6.202 ± 0.148 | 103.4 |
| 8 | 6.699 | 7.008 ± 0.162 | 104.61 |

In conclusion, the method for rapidly detecting S. typhimurium in milk through the Raman spectrum based on heavy water marking of the invention is short in detection time and high in sensitivity and accuracy, the interference from the matrix in a milk sample is small, and the method can serve as a novel method for rapidly detecting S. typhimurium in food.

It should be understood that the above preferred embodiments are only used for illustrating the content of the invention, other embodiments of the invention are available besides the above preferred embodiments, and all technical schemes formed by equivalent substitutes or equivalent transformations made by those skilled in the art based on the content and technical inspiration of the invention are within the protection scope of the invention.

1. A method for rapidly detecting S. typhimurium in milk through a Raman spectrum based on heavy water marking, characterized by comprising the steps of adding to-be-detected milk possibly containing salmonella into culture media containing heavy water for culturing, then collecting cultured salmonella cells and conducting Raman spectrum scanning, and analyzing and processing an obtained original Raman spectrum, so that S. typhimurium in the to-be-detected milk is detected.

2. The method according to claim 1, characterized in that if S. typhimurium exists in the to-be-detected milk, a CD peak appears in the corresponding original Raman spectrum within the range of 2040 cm⁻¹-2300 cm⁻¹.

3. The method according to claim 1, characterized by comprising the steps of:
(1) adding a series of S. typhimurium seed solutions with different concentrations into the culture media containing heavy water for culturing for the log phase, then collecting S. typhimurium cells at different time points respectively, conducting Raman spectrum scanning, and analyzing and processing an obtained Raman spectrum, so that a standard curve of CD/(CD+CH) % and the log value of the original S. typhimurium concentration is obtained, wherein CD/(CD+CH) % is the ratio of the peak height of the CD peak to the sum of the peak heights of the CD peak and the CH peak in the Raman spectrum;
(2) adding the to-be-detected milk possibly containing S. typhimurium into the culture media containing heavy water for culturing for the log phase, then collecting S. typhimurium cells at different time points, conducting Raman spectrum scanning, and analyzing and processing an obtained Raman spectrum and comparing the obtained Raman spectrum with the standard curve, so that the concentration of S. typhimurium in the to-be-detected milk is measured.

4. The method according to claim 3, characterized in that the step (2) further comprises the sub-step of setting operating parameters of a laser Raman spectrometer, wherein the operating parameters include the wavelength of exciting light, the laser power and the scanning time; preferably, the wavelength of the exciting light is set to 532 nm, the laser power is set to 8 mw, and the scanning time is set to 10 s.

5. The method according to claim 1, characterized in that the culturing temperature is 37 DEG C., and the culturing time is 4-8 h.

6. The method according to claim 1, characterized in that the S. typhimurium detection limit of the method is 10⁴-10⁸ cfu/m L.

7. The method according to claim 3, characterized in that in the standard curve, CD/(CD+CH) % is in the linear relation with the Log value of the original S. typhimurium concentration.

8. The method according to claim 3, characterized in that the step (2) comprises the sub-step of analyzing and processing the obtained Raman spectrum and substituting the obtained CD/(CD+CH) % to the standard curve obtained in the step (1), so that the added recovery is calculated.

9. The method according to claim 1, characterized in that the content of heavy water in the culture media containing heavy water is 0˜100 wt % and preferably is 50 wt %.

10. The method according to claim 3, characterized in that a preparation method of the S. typhimurium seed solutions comprises the steps of placing S. typhimurium in sterilized ultrapure water culture media for activation, and obtaining the S. typhimurium seed solutions at the later stage in the process of culturing the S. typhimurium for the log phase.
